(19) United States
(12) Patent Application Publication
(10) Pub. No.: US 2009/0012445 A1
(43) Pub. Date: Jan. 8, 2009

(54) Device and method for In-Situ tissue regeneration

(76) Inventor: Henryk Malak, Ellicott City, MD (US)

Correspondence Address: Henryk Malak, 8444 High Ridge Road, Ellicott City, MD, 21043, US

(21) Appl. No.: 11/824,724

(22) Filed: Jul. 3, 2007

(51) Int. Cl. A61N 1/30; A61N 1/00; A61N 2/00; A61N 5/00; A61N 7/00

(52) U.S. Cl. 604/20; 600/9; 601/1; 604/522; 606/2; 606/33; 607/2

(57) ABSTRACT

This invention describes a novel method and a novel device for In-Situ replacement of abnormal biomaterials with regenerated new biomaterials having preserved its original functionalities. For example, a scalp tissue with lost hair cells is replaced with a new regenerated scalp tissue having hair cells. The device comprising of an intrusive means to remove or destroy abnormal biomaterials from a body of an individual and a regenerative means to stimulate function-recognitive biomaterials in the body in places where was removed abnormal biomaterials, and then use the stimulated function-recognitive biomaterials to enable or to accelerate regeneration processes of new normally functional biomaterials. The described In-Situ biomaterial regeneration is performed without need of the use of the function-recognitive biomaterials harvested from other places in the body than from the places where were removed abnormal biomaterials.

### CROSS REFERENCE TO RELATED PATENT APPLICATION

This application claims the benefit of U.S. Provisional Application Ser. No. ______ filed Jun. 22, 2007, entitled “Device and Method for Tissue Regeneration”, which is incorporated by reference.

### FIELD OF THE INVENTION

This invention relates to medical and cosmetic devices and methods, and, in particular, to devices and methods for In-Situ tissue regeneration.

### BACKGROUND OF THE INVENTION

It has been for a long time scientific discussions about capabilities of a human body to regenerate tissues and organs lost due to attrition or injuries. Reports indicate that the human body has limited capabilities to regenerate lost tissues and organs, particularly to regenerate tissues and organs with preserved its original functionalities. Recent advances in stem cell research show big progress toward regeneration of tissues or organs. But so far, the regenerations are very often associated with serious medical interventions to the body, like injection stems cells to heart to rebuilt heart or to make channels in heart for revascularization of heart and increasing blood flow. Usually, the stems cells used in these procedures are harvested from specific places in the body, like bone marrow, and than these cells have to be In-Vitro modified by many transformation stages to obtain needed progenitor properties of specific organs/tissue and than are injected to the body to regenerate tissue or organs.

Recent reports also showing the presence of progenitor biomaterials in other places in the body than in bone marrow, which indicate that these progenitor biomaterials can be used for tissue regeneration in places where they there are. Some scientific reports also showing that progenitor cells can be made from fat cells, which are abundant in the body. So, there is a great need for methods and devices that will use existing progenitor cells in the body for localized-In Situ regeneration of the tissue/organs without need of use externally harvested stem cells or transplants. This present invention describes a method and a device for In-Situ regeneration tissues and organs, the regeneration with preserved tissues and organs original functionalities.

### SUMMARY OF THE INVENTION

This invention describes a novel method and a novel device for a localized (In-Situ) tissue replacement in a body without using externally harvested stem cells, or transplants. The method is based upon In-Situ removal of an abnormal tissue from a body of an individual by an intrusive means that creates an incision in the body and then the method proposes to use a noninvasive/semi-invasive regenerative means to stimulate in the incision progenitor cells arid other biomaterials to enable or to accelerate regeneration a new tissue with preserved its original functionalities. For example, in the proposed invention an abnormal hair tissue without hair cells is replaced with a new regenerated tissue that has healthy hair cells capable of growing hair.

### DESCRIPTION OF PREFERRED EMBODIMENTS

All patents, patent applications, and literatures cited or referenced in this description are incorporated herein by reference in their entireties. In the case of inconsistencies, the present disclosure, including definitions and usage, will control.

This invention describes a novel method and a novel device for In-Situ replacement of abnormal biomaterials with regenerated new biomaterials having preserved its original functionalities. For example, a scalp tissue with lost hair cells is replaced with a new regenerated scalp tissue having hair cells capable of growing hair. The proposed method comprising several steps of: providing an invasive intrusive means for removal or destroying tissue and a noninvasive/semi-invasive regenerative means for stimulation progenitor cells, making incisions in the body of an individual by the intrusive means to remove or destroy abnormal tissues, and exposing the incisions by the regenerative means to stimulate progenitor cells and other biomaterials in the incisions for a purpose of regeneration fully functional new tissues in the incisions. The described In-Situ tissue regeneration is performed without need of the use function-recognitive biomaterials harvested from other places in the body or without need of the use transplants. The proposed use of regenerative means over a natural healing means for tissue regeneration is essential in proposed invention. The use of the natural healing means not always leads to the fully functional regenerated new tissues due to following several reasons: it takes few months to regenerate tissue, the function-recognitive biomaterials present in the incision are very slow growing biomaterials, and lastly, infection risks in the incisions are high, and any infection in the incision may impede or unable the tissue regeneration. The regenerative means used in the invention are noninvasive or semi-invasive to the incisions and are capable very effectively stimulate the function-recognitive biomaterials in the incisions by transforming them from latent stages to active stages. In the proposed method the function recognitive biomaterials are progenitor cells, but the invention does not exclude other biomaterials like stems cells, neuronal cells, or other biomaterials contributing to regeneration of the fully functional tissue. The proposed step of using the intrusive means to remove or destroy the abnormal biomaterials is also essential in the proposed invention. Any presence of the abnormal biomaterials in the incisions would misinform the function-recognitive biomaterials in the incisions how to correctly regenerate the new tissues with fully functional properties.

The invention describes also the device for In-Situ replacement of abnormal tissues with regenerated fully functional tissues. The device comprising an intrusive means capable of making an incision in the body of an individual by removing or destroying an abnormal tissue that lost its original functionality, a regenerative means capable of regeneration a new tissue with recovered its original functionality by activating or stimulating the function recognitive biomaterials in the incision, and electronics administrating the intrusive means and the regenerative means. The proposed intrusive means can be a manual intrusive means or a powered intrusive means. The regenerative means in the proposed invention are noninvasive means or semi-invasive means to the incision. The regenerative means is a single energy source or a plurality energy sources selected from the group of: electromagnetic, acoustic, electric, and magnetic. The proposed device can be a medical device or a cosmetic device. The device can be a portable device or stand-alone.

Another embodiment of the invention proposes that the method can be further supported by external biomaterials harvested from different places of the body of the individual or obtained by donors, and next the external biomaterials are embedded to the incisions to accelerate regeneration of tissues in the incisions.

The proposed method is also further extended to adjacent areas of the incisions, by exposing the regenerative means to these areas that lead to expanded regeneration of tissues from the incisions to the adjacent areas.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A device for In-Situ replacement of abnormal tissues with regenerated normal tissues comprising of: an intrusive means capable of making an incision in a body of an individual by removing or destroying an abnormal tissue; a regenerative means capable of stimulating progenitor cells in the incision for the purpose of enabling or accelerating regeneration of a new normal tissue; and an electronic means for administrating the intrusive means and the regenerative means.

2. A device as in claim 1, wherein the device is a medical device or a cosmetic device.

3. A device as in claim 1, wherein the regenerative means is a single energy source or a plurality energy sources selected from the group of: electromagnetic, acoustic, mechanical, electric, and magnetic.

4. A device as in claim 3, wherein the electromagnetic source is a laser, light emitting diode, lamp, sun, far infrared source, X-Ray source, or microwave source.

5. A device as in claim 1, wherein the intrusive means is a mechanical means, an electromagnetic means, or a thermal means.

6. A device as in claim 1, wherein the electronic means comprising at least electronics connected to the intrusive means or the regenerative means.

7. A method for In-Situ replacement of abnormal tissues with regenerated normal tissues comprising steps of: providing an individual with an abnormal tissue in a body; providing an intrusive means capable of making an incision with removing or destroying the abnormal tissue in the body; providing a regenerative means capable of stimulating progenitor cells in the incision for the purpose of enabling or accelerating regeneration of a new normal tissue; making by the intrusive means the incision with removal or destroying the abnormal tissue in the body; and exposing the incision by the regenerative means to regenerate the new normal tissue in the incision.

8. A method as in claim 7, wherein the method applies to a medical procedure or to a cosmetic procedure.

9. A method as in claim 7, wherein the regenerative means is a single energy source or a plurality energy sources selected from the group of: electromagnetic, acoustic, mechanical, electric, and magnetic.

10. A method as in claim 9, wherein the electromagnetic source emits a single wavelength or multiple wavelengths with in a wavelength range of 0.2 micrometer to 20,000 microns.

11. A method as in claim 7, wherein the intrusive means is a mechanical means, an electromagnetic means, or a thermal means.

12. A method as in claim 7, wherein the regeneration of the new tissue in the incision is further accelerated with an external biomaterial embedded into the incision.

13. A method as in claim 7, wherein the regenerative means is additionally exposed to a surrounding area of the incision to extend the regeneration of the new tissue from the incision into the surrounding area of the incision.
